SEQUENCE LISTING (5 sequences)

SEQ ID NO: 1 - DNA, 1740 bp, Corynebacterium glutamicum
  CDS (341)..(1495)
gtattgcgccgatttcccagattttgattgaaaccgatgcgccgtatatgacgccggagccgtttcgggggagtaggaatgagccgtcgttgattggtcatacggcgctatgcattgcggaggttcgggggatggctgtggaggatgttgcggcggctttgaatgagaattttgatcgcgtttatggggtcacaaatctataacgtgaggtagctcacagtcaatctgttggccgtggtcagctgtgggggttgtggtgggtgtgactgaagtttatgaagttgcacgccacggcgttttggtgatggacgggggtagtttgttaccgtattgtgactaattgttaattcccccgagagcgaagaagttttacatggcgccccatcagaagtcacggatcaaccggatcaacagcacccgctcggtgccgttgcgtttggctaccggtggcgtgctcgccaccttgcttatcggcggagtcaccgctgcagctaccaaaaaggacatcattgttgatgtcaacggcgagcagatgtccctagtgactatgtccggcactgttgaaggtgtgctggcgcaagctggtgtggaacttggtgaccaggacattgtttccccttcactggattcatccatcagtgatgaagacactgtgactgttcgtactgccaagcaggtggcgctcgtggtggaaggtcaaatccaaaacgtgaccaccactgcggtttccgtggaggacctcctgcaggaagtcggtggcattaccggtgctgatgcggtggacgctgatctttcagagaccatcccagaatctggtttgaaggtgagtgttaccaagccgaagattatttccatcaatgatggtggcaaggtcacttacgtttctttggcagctcagaacgtacaggaagccctagagctgcgggatattgagctgggtgctcaggaccgcattaatgtgcctctggatcagcagctgaagaacaacgctgcgatccagatcgaccgcgttgacaacaccgaaatcactgaaactgtgtctttcgatgctgagccaacctacgtggatgatccagaagctccagctggcgatgaaactgtggtcgaagaaggcgctcctggaaccaaggaagttactcgcaccgtaacaaccgttaatggtcaggaagaatcttccacggtgatcaatgaagttgaaatcaccgcagcaaagccagcaaccattagccgtggcaccaaaactgtcgctgcaaactccgtgtgggatcagctggcacagtgtgaatccggcggaaactgggcaatcaacacaggtaatggcttctccggcggcctacagttccacccacagacctggctcgcatacggtggtggagctttctccggtgacgcttccggtgcaagccgtgaacagcaaatctccatcgcagaaaaggttcaggctgcacaaggttggggagcatggcctgcttgcaccgcaagcttgggcatccgatagtagaaatctggcatccaataggtagattgggatgctatggaagaaccctcaggtgcacagctgctcggcccggtagaaatccgtgcgctggcagaaaagctcgacgtcacaccaactaagaagttggggcagaactttgttcacgatcccaacacggtgcgtcgcattgttgctgcggcagagctcaccccagacgaccacgtggtggaagttggccctggtctgggctctctgacccttgc

SEQ ID NO: 2 - protein, 385 aa, Corynebacterium glutamicum
LLIPPRAKKFYMAPHQKSRINRINSTRSVPLRLATGGVLATLLIGGVTAAATKKDIIVDVNGEQMSLVTMSGTVEGVLAQAGVELGDQDIVSPSLDSSISDEDTVTVRTAKQVALVVEGQIQNVTTTAVSVEDLLQEVGGITGADAVDADLSETIPESGLKVSVTKPKIISINDGGKVTYVSLAAQNVQEALELRDIELGAQDRINVPLDQQLKNNAAIQIDRVDNTEITETVSFDAEPTYVDDPEAPAGDETVVEEGAPGTKEVTRTVTTVNGQEESSTVINEVEITAAKPATISRGTKTVAANSVWDQLAQCESGGNWAINTGNGFSGGLQFHPQTWLAYGGGAFSGDASGASREQQISIAEKVQAAQGWGAWPACTASLGIR

SEQ ID NO: 3 - DNA, 629 bp, Corynebacterium glutamicum
ggaacttggtgaccaggacattgtttccccttcactggattcatccatcagtgatgaagacactgtgactgttcgtactgccaagcaggtggcgctcgtggtggaaggtcaaatccaaaacgtgaccaccactgcggtttccgtggaggacctcctgcaggaagtcggtggcattaccggtgctgatgcggtggacgctgatctttcagagaccatcccagaatctggtttgaaggtgagtgttaccaagccgaagattatttccatcaatgatggtggcaaggtcacttacgtttctttggcagctcagaacgtacaggaagccctagagctgcgggatattgagctgggtgctcaggaccgcattaatgtgcctctggatcagcagctgaagaacaacgctgcgatccagatcgaccgcgttgacaacaccgaaatcactgaaactgtgtctttcgatgctgagccaacctacgtggatgatccagaagctccagctggcgatgaaactgtggtcgaagaaggcgctcctggaaccaaggaagttactcgcaccgtaacaaccgttaatggtcaggaagaatcttccacggtgatcaatgaagttgaaatcaccgcagcaaagccag

SEQ ID NO: 4 - DNA, 20 bp, PCR primer
ggaacttggtgaccaggaca

SEQ ID NO: 5 - DNA, 20 bp, PCR primer
ctggctttgctgcggtgatt

(19) United States
(12) Patent Application Publication
(10) Pub. No.: US 2002/0106748 A1
(43) Pub. Date: Aug. 8, 2002

(54) Novel nucleotide sequences encoding the zwa2 gene

(76) Inventors: Bettina Mockel, Dusseldorf (DE); Anke Weissenborn, Tubingen (DE); Walter Pfefferle, Halle (DE); Achim Marx, Bielefeld (DE); Alfred Puhler, Bielefeld (DE); Jorn Kalinowski, Bielefeld (DE); Brigitte Bathe, Salzkotten (DE); Nicole Dusch, Bielefeld (DE)

Correspondence Address: PILLSBURY WINTHROP LLP, 9TH FLOOR, 1100 NEW YORK AVENUE, N.W., WASHINGTON, DC, 20005, US

(21) Appl. No.: 09/733,386

(22) Filed: Dec. 4, 2000

(30) Foreign Application Priority Data
Dec. 9, 1999 (DE) 199 59 3427.2

(51) Int. Cl. C12P 13/04; C12P 13/08; C07H 21/04; C12N 1/21

(52) U.S. Cl. 435/106; 435/115; 435/252.3; 435/69.1; 536/23.2

(57) ABSTRACT

The invention provides novel isolated polynucleotides containing a polynucleotide sequence chosen from the group

a) a polynucleotide which is at least 70% identical to a polynucleotide which encodes a polypeptide which contains the amino acid sequence SEQ ID NO:2,

b) a polynucleotide which encodes a polypeptide which contains an amino acid sequence which is at least 70% identical to the amino acid sequence SEQ ID NO:2,

c) a polynucleotide which is complementary to the polynucleotides in a) or b), and

d) a polynucleotide containing at least 15 nucleotides in sequence from the polynucleotide sequence in a), b) or c),

and a process for the fermentative preparation of L-lysine with attenuation of the zwa2 gene in the coryneform bacteria used.

This application claims priority from German Application No. 199 59 327.2, filed on Dec. 9, 1999, the subject matter of which is hereby incorporated herein by reference.

### BACKGROUND OF THE INVENTION

1. Field of the Invention

The invention provides nucleotide sequences encoding the zwa2 gene and a process for fermentative preparation of amino acids, in particular L-lysine, using coryneform bacteria in which the zwa2 gene is attenuated.

2. Background Information

Amino acids, in particular L-lysine, are used in human medicine and in the pharmaceutical industry, but especially in the animal nutrition sector.

It is known that amino acids can be prepared by fermenting strains of coryneform bacteria, in particular Corynebacterium glutamicum. Due to the great importance of these processes, work relating to improving the methods of manufacture is always in progress. Process improvements may relate to fermentation technology measures such as, for example, stirring and supplying with oxygen, or the composition of the nutrient media such as, for example, the sugar concentration during fermentation, or working up to full product status by, for example, ion exchange chromatography, or the intrinsic performance characteristics of the microorganism itself.

To improve the performance characteristics of these microorganisms, the methods of mutagenesis, selection and mutant choice are applied. Strains which are resistant to antimetabolites, such as, for example, the lysine analogon S-(2-aminoethyl)-cysteine, or are auxotrophic for regulatorily important metabolites and which produce L-amino acids are obtained in this way.

For some years, the methods of recombinant DNA technology have also been used for the strain improvement of amino acid-producing strains of Corynebacterium.

### SUMMARY OF THE INVENTION

### Object of the Invention

It is an object of the invention to provide novel means for the improved fermentative preparation of amino acids, in particular L-lysine.

### Description of the Invention

L-amino acids, in particular L-lysine are used in human medicine, in the pharmaceutical industry and in particular in animal nutrition. Thus there is general interest in providing new improved processes for preparing amino acids, in particular L-lysine.

Whenever L-lysine or lysine is mentioned herein, it is intended to include not only the bases but also the salts such as, for example, lysine monochloride or lysine sulfate.

The invention provides an isolated polynucleotide from coryneform bacteria containing a polynucleotide sequence chosen from the group

a) a polynucleotide which is at least 70% identical to a polynucleotide which encodes a polypeptide that contains the amino acid sequence SEQ ID NO:2,

b) a polynucleotide which encodes a polypeptide which contains an amino acid sequence that is at least 70% identical to the amino acid sequence SEQ ID NO:2,

c) a polynucleotide which is complementary to the polynucleotides in a) or b), and

d) a polynucleotide containing at least 15 nucleotides in sequence from the polynucleotide sequence in a), b) or c).

The invention also provides a polynucleotide with the above features that is preferably a replicatable DNA, containing:

(i) the nucleotide sequence, shown in SEQ ID NO:1, which codes for the zwa2 gene,

(ii) at least one sequence which corresponds to sequence (i) within the scope of degeneration of the genetic code or,

(iii) at least one sequence which hybridises with sequences complementary to sequences (i) or (ii), and optionally

(iv) functionally neutral sense mutations in (i).

Also provided are

a polynucleotide as described above, containing the nucleotide sequence as represented in SEQ ID NO:1,

a vector, containing the polynucleotide with the features of d), as detailed above, in particular pCR2.1zwa2int, deposited in E.coli DSM 13113

and coryneform bacteria acting as host cells which are obtained by integration mutagenesis with this vector.

The invention also provides polynucleotides which consist substantially of one polynucleotide sequence which are obtainable by screening by means of hybridising a corresponding gene library which contains the complete gene with the polynucleotide sequence corresponding to SEQ ID NO:1 or a portion thereof, using a probe which contains the sequence of the polynucleotide in accordance with SEQ ID NO:1 described hereinabove or a fragment thereof, and isolating the DNA sequence mentioned.

Polynucleotide sequences in accordance with the invention are suitable as hybridisation probes for RNA, cDNA and DNA, in order to isolate the full length of cDNA which encodes the Zwa2 gene product and in order to isolate those product cDNAs or genes which are very similar to the sequence with the zwa2 gene.

Polynucleotide sequences in accordance with the invention are also suitable for use as primers with the aid of which DNA can be produced, using the polymerase chain reaction (PCR), from genes which code for the zwa2 gene.

Those oligonucleotides which can be used as probes or primers contain at least 30, preferably at least 20, very particularly preferably at least 15 nucleotides in sequence. Oligonucleotides with a length of at least 40 or 50 nucleotides are also suitable.

“Isolated” means being taken out of its natural surroundings.

“Polynucleotide” refers in general to polyribonucleotides and polydeoxyribonucleotides, wherein they may be non-modified RNA or DNA or modified RNA or DNA.

“Polypeptides” are understood to be peptides or proteins which contain two or more amino acids linked via peptide bonds.

Polypeptides in accordance with the invention include polypeptides in accordance with SEQ ID NO:2, in particular those with the biological activity of the gene product from the zwa2 gene and also those which are at least 70% identical to the polypeptide in accordance with SEQ ID NO:2, preferably being at least 80% and in particular at least 90% to 95% identical to the polypeptide in accordance with SEQ ID NO:2 and which have the activity mentioned.

The invention also provides a process for the fermentative preparation of amino acids, in particular L-lysine, using coryneform bacteria which in particular already produce the amino acid and in which the nucleotide sequences encoding the zwa2 gene are attenuated, in particular expressed at a low level.

The microorganisms which are provided by the present invention can produce L-lysine from glucose, saccharose, lactose, fructose, maltose, molasses, starch, cellulose or from glycerol and ethanol. They may be representatives of coryneform bacteria in particular of the genus Corynebacterium. From the genus Corynebacterium, the species Corynebacterium glutamicum should be mentioned in particular, this being known in the specialist field for its ability to produce L-amino acids.

Suitable strains of the genus Corynebacterium, in particular the species Corynebacterium glutamicum, are, for example, the known wild type strains

Corynebacterium glutamicum ATCC13032

Corynebacterium acetoglutamicum ATCC15806

Corynebacterium acetoacidophilum ATCC13870

Corynebacterium melassecoloa ATCC17965

Corynebacterium thermoaminogenes FERM BP-1539

Brevibacterium flavum ATCC14067

Brevibacterium lactofermentum ATCC13869 and

Brevibacterium divaricatum ATCC14020

and L-lysine producing mutants or strains produced therefrom such as, for example

Corynebacterium glutamicum FERM-P 1709

Brevibacterium flavum FERM-P 1708

Brevibacterium lactofermentum FERM-P 1712

Corynebacterium glutamicum FERM-P 6463

Corynebacterium glutamicum FERM-P 6464 and

Corynebacterium glutamicum DSM5715

The inventors have succeeded in isolating from C. glutamicum the novel zwa2 gene coding for the Zwa2 gene product.

In order to isolate the zwa2 gene or any other genes from C. glutamicum a gene library from this microorganism is first constructed in E. coli. The construction of gene libraries is described in generally known textbooks and manuals. As examples, the textbook by Winnacker: Gene und Klone, Eine Einführung in die Gentechnologie (Verlag Chemie, Weinheim, Germany, 1990) or the manual by Sambrook et al.: Molecular Cloning, A Laboratory Manual (Cold Spring Harbor Laboratory Press, 1989) may be mentioned. A very well-known gene library is that from the E. coli K-12 strain W3110, which was compiled by Kohara et al. (Cell 50, 495-508 (1987)) in λ-vectors. Bathe et al. (Molecular and General Genetics, 252:255-265, 1996) describe a gene library from C. glutamicum ATCC13032, which was constructed with the aid of the cosmid vector SuperCos I (Wahl et al., 1987, Proceedings of the National Academy of Sciences USA, 84:2160-2164) in E. coli K-12 strain NM554 (Raleigh et al., 1988, Nucleic Acids Research 16:1563-1575). Börmann et al. (Molecular Microbiology 6(3), 317-326 (1992)) also describe a gene library from C. glutamicum ATCC13032 using the cosmid pHC79 (Hohn and Collins, Gene 11, 291-298 (1980)). To prepare a gene library from C. glutamicum in E. coli, plasmids such as pBR322 (Bolivar, Life Sciences, 25, 807-818 (1979)) or pUC9 (Vieira et al., 1982, Gene, 19: 259-268) can also be used. E. coli strains which are especially suitable as hosts are those which are restriction and recombination defective. An example of these is the strain DH5αmcr, which was described by Grant et al. (Proceedings of the National Academy of Sciences USA, 87 (1990) 4645-4649). The long DNA fragments cloned with the aid of cosmids may then be subcloned in commonly used vectors suitable for sequencing and then sequenced, as described, for example, in Sanger et al. (Proceedings of the National Academy of Sciences of the United States of America, 74:5463-5467, 1977).

The new DNA sequence encoding the zwa2 gene was obtained in this way, and this is a constituent of the present invention as SEQ ID NO:1. Furthermore, the amino acid sequence of the zwa2 gene in the corresponding gene product was derived from the available DNA sequence. The amino acid sequence of the Zwa2 gene product being produced is shown in SEQ ID NO:2.

Coding DNA sequences which are produced from SEQ ID NO:1 due to the degeneracy of the genetic code are also included in the invention. Similarly, DNA sequences which hybridise with SEQ ID NO:1 or portions of SEQ ID NO:1 are also included in the invention. Finally, DNA sequences which are prepared by the polymerase chain reaction (PCR) using primers which are produced from SEQ ID NO:1 are also included in the invention.

A person skilled in the art will find instructions for identifying DNA sequences by means of hybridisation, inter alia, in the manual “The DIG System Users Guide for Filter Hybridization” produced by Boehringer Mannheim GmbH (Mannheim, Germany, 1993) and in Liebl et al. (International Journal of Systematic Bacteriology (1991) 41: 255-260). A person skilled in the art will find instructions for amplifying DNA sequences with the aid of the polymerase chain reaction (PCR), inter alia, in the manual by Gait: Oligonucleotide synthesis: a practical approach (IRL Press, Oxford, UK, 1984) and in Newton and Graham: PCR (Spektrum Akademischer Verlag, Heidelberg, Germany, 1994).

The inventors discovered that coryneform bacteria produce amino acids, in particular L-lysine, in an improved manner after attenuation of the zwa2 gene.

To produce attenuation, either the expression of the zwa2 gene or the catalytic properties of the enzyme protein can be reduced or switched off. Optionally, both measures can be combined.

Gene expression can be reduced by suitable culture management or by genetic modification (mutation) of the signal structures for gene expression. Signal structures for gene expression are, for example, repressor genes, activator genes, operators, promoters, attenuators, ribosome bonding sites, the start codon and terminators. Data on these may be found by a person skilled in the art, for example, in patent application WO 96/15246, in Boyd and Murphy (Journal of Bacteriology 170: 5949 (1988)), in Voskuil and Chambliss (Nucleic Acids Research 26: 3548 (1998), in Jensen and Hammer (Biotechnology and Bioengineering 58: 191 (1998)), in Patek et al. (Microbiology 142: 1297 (1996)) and in standard textbooks on genetics and molecular biology such as, for example, the textbook by Knippers (“Molekulare Genetik”, 6th edition, Georg Thieme Verlag, Stuttgart, Germany, 1995) or the textbook by Winnacker (“Gene und Klone”, VCH Verlagsgesellschaft, Weinheim, Germany, 1990).

Mutations which lead to a change or reduction in the catalytic properties of enzyme proteins are known from the prior art; the articles by Qiu and Goodman (Journal of Biological Chemistry 272: 8611-8617 (1997)), Sugimoto et al. (Bioscience Biotechnology and Biochemistry 61: 1760-1762 (1997)) and Mockel (“Die Threonindehydratase aus Corynebacterium glutamicum: Aufhebung der allosterischen Regulation und Struktur des Enzyms”, Reports from the Jülich Research Centre, Jül-2906, ISSN09442952, Jülich, Germany, 1994) may be mentioned as examples. Brief reviews can be found in standard textbooks on genetics and molecular biology such as, for example, the textbook by Hagemann (“Allgemeine Genetik”, Gustav Fischer Verlag, Stuttgart, 1986).

Transitions, transversions, insertions and deletions are considered to be mutations. Mis-sense mutations or non-sense mutations are referred to, depending on the effect of amino acid exchange on the enzyme activity. Insertions or deletions of at least one base pair in a gene lead to frame shift mutations as a result of which the wrong amino acids are incorporated or translation is terminated prematurely. Deletions of several codons typically leads to complete loss of enzyme activity. Instructions for producing these types of mutations are part of the prior art and can be found in standard textbooks on genetics and molecular biology such as, for example, the textbook by Knippers (“Molekulare Genetik”, 6th edition, Georg Thieme Verlag, Stuttgart, Germany, 1995), the textbook by Winnacker (“Gene und Klone”, VCH Verlagsgesellschaft, Weinheim, Germany, 1990) or the textbook by Hagemann (“Allgemeine Genetik”, Gustav Fischer Verlag, Stuttgart, 1986).

An example of a plasmid with the aid of which insertion mutagenesis of the zwa2 gene can be performed is pCR2.1zwa2int (FIG. 1).

Plasmid pCR2.1zwa2int consists of the plasmid pCR2.1-TOPO described by Mead at al. (Bio/Technology 9:657-663 (1991)), into which an internal fragment of the zwa2 gene shown in SEQ ID NO:3 has been incorporated. This plasmid leads to a total loss of function after transformation and homologous recombination in the chromosomal zwa2 gene (insertion). The strain DSM5715::pCR2.1zwa2int in which the zwa2 gene is switched off was prepared, for example, in this way. Further instructions for and explanations of insertion mutagenesis can be found, for example, in Schwarzer and Pühler (Bio/Technology 9,84-87 (1991)) or Fitzpatrick et al. (Applied Microbiology and Biotechnology 42, 575-580 (1994)).

In addition, it may be advantageous for the production of L-amino acids, in particular L-lysine, as well as attenuating the zwa-2 gene, to enhance, in particular to overexpress, one or more enzymes in the relevant biosynthetic pathway, glycolysis, anaplerotic reactions, the citric acid cycle or amino acid export.

Thus, for example, for the production of L-lysine, one or more of the genes chosen from the group

the dapA gene encoding dihydrodipicolinate synthase (EP-B 0 197 335),

the dapD gene encoding tetradihydrodipicolinate succinylase (Wehrmann et al., Journal of Bacteriology 180, 3159-3165 (1998)),

the lysc gene encoding a feed back resistant aspartate kinase,

the dapE gene encoding succinyldiaminopimelate desuccinylase (Wehrmann et al., Journal of Bacteriology 177: 5991-5993 (1995)),

the gap gene encoding glyceraldehyde-3-phosphate dehydrogenase (Eikmanns (1992), Journal of Bacteriology 174:6076-6086),

the pyc gene encoding pyruvate carboxylase (DE-A-198 31 609),

the mqo gene encoding malate:quinone oxidoreductase (Molenaar et al., European Journal of Biochemistry 254, 395-403 (1998)),

the lysE gene encoding lysine export (DE-A-195 48 222) can be simultaneously enhanced, in particular overexpressed or amplified.

Furthermore, it may be advantageous for the production of amino acids, in particular L-lysine, simultaneously to attenuate, in addition to the zwa2 gene,

the gene encoding phosphate pyruvate carboxykinase (DE 199 50 409.1; DSM 13047) and/or

the pgi gene encoding glucose-6-phosphate isomerase (US 09/396,478; DSM 12969).

Furthermore, it may also be advantageous for the production of amino acids, in particular L-lysine, in addition to attenuating the zwa2 gene, to switch off unwanted secondary reactions (Nakayama: “Breeding of Amino Acid Producing Micro-organisms”, in: Overproduction of Microbial Products, Krumphanzl, Sikyta, Vanek (eds.), Academic Press, London, UK, 1982).

The microorganisms containing the polynucleotide with features a)-d) are also provided by the invention and may be cultivated continuously or batchwise in a batch process or a fed batch process or a repeated fed batch process for the purposes of producing amino acids, in particular L-lysine. A summary of known cultivation methods is described in the textbook by Chmiel (Bioprozesstechnik 1. Einfuhrung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)).

The culture medium to be used has to satisfy the requirements of the particular strains in a suitable manner. Descriptions of culture media for different microorganisms are given in the manual “Manual of Methods for General Bacteriology” by the American Society for Bacteriology (Washington D.C., USA, 1981). Sources of carbon which may be used are sugar and carbohydrates such as e.g. glucose, saccharose, lactose, fructose, maltose, molasses, starch and cellulose, oils and fats such as e.g. soya oil, sunflower oil, groundnut oil and coconut butter, fatty acids such as e.g. palmitic acid, stearic acid and linoleic acid, alcohols such as, for example, glycerol and ethanol and organic acids such as, for example, acetic acid. These substances may be used individually or as a mixture. Sources of nitrogen which may be used are organic nitrogen-containing compounds such as peptones, yeast extract, meat extract, malt extract, corn steep liquor, soya bean meal and urea or inorganic compounds such as ammonium sulfate, ammonium chloride, ammonium phosphate, ammonium carbonate and ammonium nitrate. The sources of nitrogen may be used individually or as a mixture. Sources of phosphorus which may be used are phosphoric acid, potassium dihydrogen phosphate or dipotassium hydrogen phosphate or the corresponding sodium-containing salts. The culture medium also has to contain salts of metals, such as, for example, magnesium sulfate or iron sulfate, which are needed for growth purposes. Finally, essential growth substances such as amino acids and vitamins can be used in addition to the substances mentioned above. In addition to this, suitable precursors may be added to the culture medium. The feed substances mentioned can be added to the culture in the form of a single mixture or may be supplied gradually in an appropriate manner during cultivation.

To regulate the pH of the culture, basic compounds such as sodium hydroxide, potassium hydroxide, ammonia or ammonia water or acid compounds such as phosphoric acid or sulfuric acid are used in an appropriate manner. To regulate the production of foam, antifoaming agents such as, for example, polyglycol esters of fatty acids may be used. To maintain stability of the plasmids, suitable selective substances such as, for example, antibiotics may be added to the medium. In order to maintain aerobic conditions, oxygen or oxygen-containing gas mixtures such as, for example, air are passed into the culture. The temperature of the culture is normally 20° C. to 45° C. and preferably 25° C. to 40° C. The culture is cultivated until a maximum in the lysine concentration has been produced. This objective is normally reached within 10 hours to 160 hours.

Methods for determining L-amino acids are known from the prior art. Analysis can be performed as described in Spackman et al. (Analytical Chemistry, 30, (1958), 1190) using anion exchange chromatography followed by ninhydrin derivatisation or reversed phase HPLC may be used, as described in Lindroth et al. (Analytical Chemistry (1979) 51: 1167-1174).

An integration vector suitable for mutagenesis was deposited in E.coli at the German Collection for Microorganisms and Cell Cultures (DSMZ, Braunschweig, Germany) in accordance with the Budapest Treaty:

Escherichia coli strain TOP10F′/pCR2.1zwa2int as DSM 13113

In addition to attenuating the zwa2 gene, it may be advantageous to enhance the zwa1 gene or the effect of the associated Zwa1 gene product. The corresponding gene and the associated measures can be found in German patent application 199 59 328.0 which was filed in parallel with this application.

An integration vector suitable for mutagenesis pCR2.1zwa1exp was deposited in E.coli DH5 under the no. DSM13115.

### BRIEF DESCRIPTION OF THE DRAWING

FIG. 1: Map of the plasmid pCR2.1zwa2int

The data relating to length are understood to be approximate values.

The abbreviations and names used have the following meaning.

| | |
|---|---|
| ColE1 ori: | Replication origin of the plasmid ColE1 |
| lacZ: | 5′ end of the β-galactosidase gene |
| f1 ori: | Replication origin of the phage f1 |
| KanR: | Kanamycin resistance |
| ApR: | Ampicillin resistance |
| EcoRI: | Cleavage site of the restriction enzyme |
| | EcoRI |
| zwa2: | Internal fragment of the zwa2 gene |

### DETAILED DESCRIPTION OF THE INVENTION

The present invention is explained in more detail in the following, using working examples.

### EXAMPLE 1

Preparing a genomic cosmid gene library from Corynebacterium glutamicum ATCC 13032

Chromosomal DNA from Corynebacterium glutamicum ATCC 13032 was isolated in the way described in Tauch et al. (1995, Plasmid 33:168-179) and partly cleaved with the restriction enzyme Sau3AI (Amersham Pharmacia, Freiburg, Germany, product description Sau3AI, Code no. 27-0913-02). The DNA fragments were dephosphorylated with shrimp alkaline phosphatase (Roche Molecular Biochemicals, Mannheim, Germany, product description SAP, Code no. 1758250). The DNA from the cosmid vector SuperCosl (Wahl et al. (1987) Proceedings of the National Academy of Sciences U.S.A 84:2160-2164), purchased from the Stratagene Co. (La Jolla, USA, product description SuperCosl cosmid vector kit, Code no. 251301) was cleaved with the restriction enzyme XbaI (Amersham Pharmacia, Freiburg, Germany, product description XbaI, Code no. 27-0948-02) and also dephosphorylated with shrimp alkaline phosphatase. The cosmid DNA was then cleaved with restriction enzyme BamHI (Amersham Pharmacia, Freiburg, Germany, product description BamHI, Code no. 27-0868-04). The cosmid DNA treated in this way was mixed with the treated ATCC13032 DNA and the mixture was treated with T4-DNA-Ligase (Amersham Pharmacia, Freiburg, Germany, product description T4-DNA-Ligase, Code no.27-0870-04). The ligation mixture was then packaged into phages with the aid of Gigapack II XL packing extract (Stratagene, La Jolla, USA, product description Gigapack II XL packing extract, Code no. 200217). In order to infect E. coli strain NM554 (Raleigh et al. 1988, Nucleic Acid Research 16:1563-1575) the cells were taken up in 10 mM MgSO₄ and mixed with an aliquot of the phage suspension. Infection and standardisation of the cosmid library was performed as described in Sambrook et al. (1989, Molecular Cloning: A laboratory Manual, Cold Spring Harbor), wherein the cells were plated out on LB agar (Lennox, 1955, Virology, 1:190) with 100 μg/ml of ampicillin. After incubation overnight at 37° C., individual recombinant clones were selected.

### EXAMPLE 2

Isolation and sequencing of the zwa2 gene

The cosmid DNA from an individual colony was isolated using the Qiaprep spin miniprep kit (Product No. 27106, Qiagen, Hilden, Germany) in accordance with the manufacturer's data and partly cleaved with the restriction enzyme Sau3AI (Amersham Pharmacia, Freiburg, Germany, product description Sau3AI, Product No. 27-0913-02). The DNA fragments were dephosphorylated with shrimp alkaline phosphatase (Roche Molecular Biochemicals, Mannheim, Germany, produce description SAP, Product No. 1758250). After gel electrophoretic separation, the cosmid fragments in the size range 1500 to 2000 were isolated using the QiaExII gel extraction kit (Product No. 20021, Qiagen, Hilden, Germany). DNA from the sequencing vector pZero-1 purchased from the Invitrogen Co. (Groningen, the Netherlands, product description zero background cloning kit, Product No. K2500-01) was cleaved using the restriction enzyme BamHI (Amersham Pharmacia, Freiburg, Germany, product description BamHI, Product No. 27-0868-04). The cosmid fragments were ligated in the sequencing vector pZero-1 using the method described in Sambrook et al. (1989, Molecular Cloning: A laboratory Manual, Cold Spring Harbor), wherein the DNA mixture was incubated overnight with T4 ligase (Pharmacia Biotech, Freiburg, Germany). This ligation mixture was then electropored in E. coli strain DH5αMCR (Grant, 1990, Proceedings of the National Academy of Sciences U.S.A., 87:4645-4649) (Tauch et al. 1994, FEMS Microbiol Letters, 123:343-7) and plated out on LB agar (Lennox, 1955, Virology, 1:190) with 50 pg/ml zeocin. Plasmid preparation of the recombinant clones was achieved with a Biorobot 9600 (Product No. 900200, Qiagen, Hilden, Germany). Sequencing was achieved using the dideoxy-chain termination method of Sanger et al. (1977, Proceedings of the National Academy of Sciences U.S.A., 74:5463-5467) with modifications by Zimmermann et al. (1990, Nucleic Acids Research, 18:1067). The “RR dRhodamin Terminator Cycle Sequencing Kit” from PE Applied Biosystems(Product No. 403044, Weiterstadt, Germany) was used. Gel electrophoretic separation and analysis of the sequencing reaction was performed in a “Rotiphorese NF Acrylamid/Bisacrylamid” gel (29:1) (Product No. A124.1, Roth, Karlsruhe, Germany) using the “ABI Prism 377” sequencing equipment from PE Applied Biosystems (Weiterstadt, Germany).

The crude sequence data obtained were then processed using a Staden software package (1986, Nucleic Acids Research, 14:217-231) Version 97-0. The individual sequences in the pzerol derivatives were assembled to give a cohesive contig. Computer-aided coding region analyses were drawn up using the XNIP programme (Staden, 1986, Nucleic Acids Research, 14:217-231). Homology analyses were performed using the “BLAST search programs” (Altschul et al., 1997, Nucleic Acids Research, 25:3389-3402) against the non-redundant data bank at the “National Center for Biotechnology Information” (NCBI, Bethesda, Md., USA).

The nucleotide sequence obtained for the zwa2 gene is shown in SEQ ID NO:1. Analysis of the nucleotide sequence produced an open reading frame of 1740 base pairs which was called the zwa2 gene. The zwa2 gene encoded a polypeptide of 385 amino acids, which is shown in SEQ ID NO:2.

### EXAMPLE 3

Preparation of an integration vector for the insertion mutagenesis of the zwa2 gene

Chromosomal DNA from the strain ATCC 13032 was isolated by the method of Eikmanns et al. (Microbiology 140: 1817-1828 (1994)). On the basis of the sequence for the zwa2 gene disclosed for C. glutamicum in example 2, the following oligonucleotides were chosen for the polymerase chain reaction:

zwa2-in1:
5′ GGA ACT TGG TGA CCA GGA CA 3′
zwa2-in2:
5′ CTG GCT TTG CTG CGG TGA TT 3′

The primers shown were synthesised by the MWG Biotech Co. (Ebersberg, Germany) and the PCR reaction was performed using the standard PCR method of Innis et al. (PCR protocols. A guide to methods and applications, 1990, Academic Press) with Pwo polymerase from the Boehringer Co. With the aid of the polymerase chain reaction, an approximately 0.6 kb large DNA fragment was isolated, shown in SEQ ID NO:3, which included an internal fragment of the zwa2 gene.

The amplified DNA fragment was ligated with the TOPO TA cloning kit from the Invitrogen Corporation (Carlsbad, Calif., USA; catalogue number K4500-01) in vector pCR2.1-TOPO (Mead at al. (1991) Bio/Technology 9:657-663). The E. coli strain Top10F′ was electropored with the ligation mixture (Hanahan, In: DNA cloning. A practical approach. Vol.I. IRL-Press, Oxford, Washington D.C., USA). Selection of the plasmid-carrying cells was achieved by plating out the transformation batch on LB agar (Sambrook et al., Molecular cloning: a laboratory manual. 2^{nd} Ed. Cold Spring Harbor Laboratory Press, Cold Spring Harbor, N.Y.), which had been supplemented with 25 mg/l kanamycin. Plasmid DNA was isolated from one of the transformants with the aid of a QIAprep spin miniprep kit from the Qiagen Co. and tested by restriction with the restriction enzyme EcoRI followed by agarose gel electrophoresis (0.8%). The plasmid was named pCR2.1zwa2int.

### EXAMPLE 4

Integration mutagenesis of the zwa2 gene into the lysine-producer DSM 5715

The vector called pCR2.1zwa2int in example 2 was electropored in Corynebacterium glutamicum DSM 5715 using the electroporation method of Tauch et al.(FEMS Microbiological Letters, 123:343-347 (1994)). The strain DSM 5715 is an AEC-resistant lysine producer. The vector pCR2.1zwa2int cannot replicate autonomously in DSM5715 and only remains in the cells when it has integrated into the chromosome of DSM 5715. The selection of clones with pCR2.1zwa2int integrated in the chromosome was achieved by plating out the electroporation batch on LB agar (Sambrook et al., Molecular cloning: a laboratory manual. 2^{nd} Ed. Cold Spring Harbor Laboratory Press, Cold Spring Harbor, N.Y., 1989) which had been supplemented with 15 mg/l kanamycin. In order to detect integration, control PCR reactions were performed using the standard method of Innis et al. (PCR protocols. A guide to methods and applications, 1990, Academic Press) using Pwo polymerase from the Boehringer Co. By combining the primers zwa1-in1 and zwa2-in2 (see example 3) with the primers M13 universal forward (5′-gttttcccagtcacgac-3′; Invitrogen Corporation, Cat. No. N540-02) and M13 universal reverse (5′-caggaaacagctatgac-3′; Invitrogen Corporation, Cat. No. N530-02) which can only bond within the sequence of the vector pCR2.1zwa2int, it can be shown that the plasmid pCR2.1zwa2int had inserted within the chromosomal zwa2 gene in the chromosome of the lysine-producer DSM5715. The strain was called DSM5715::pCR2.1zwa2int.

### EXAMPLE 5

Preparing Lysine

The C. glutamicum strain DSM5715::pCR2.1zwa2int obtained in example 3 was cultivated in a nutrient medium suitable for the production of lysine and the lysine concentration in the culture supernatant liquid was determined.

To do this, the strain was first incubated on agar plates with the corresponding antibiotic (brain/heart agar with kanamycin (25 mg/l)) for 24 hours at 33° C. Starting from this agar plate culture, a preliminary culture was inoculated (10 ml of medium in 100 ml conical flasks). Complete medium CgIII was used as the medium for the preliminary culture. Kanamycin (25 mg/l) was added to this. The preliminary culture was incubated for 48 hours at 33° C. 240 rpm on a shaker. A main culture was inoculated with this preliminary culture so that the initial OD (660 nm) of the main culture was 0.1. The medium MM was used for the main culture.

| Medium MM | | |
|---|---|---|
| CSL (Corn Steep Liquor) | 5 | g/l |
| MOPS | 20 | g/l |
| Glucose (autoclaved separately) | 50 | g/l |
| Salts: | | |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (filtered sterile) | 0.3 | mg/l |
| Thiamin * HCl (filtered sterile) | 0.2 | mg/l |
| Leucine (filtered sterile) | 0.1 | g/l |
| CaCO₃ | 25 | g/l |

CSL, MOPS and the salt solution are adjusted to pH 7 with ammonia water and autoclaved. Then the sterile substrate and vitamin solutions are added, as well as the dry autoclaved CaCO₃.

Cultivation was performed in 10 ml volumes in a 100 ml conical flask with baffles. Kanamycin (25 mg/l) was added. Cultivation was performed at 33° C. and 80% atmospheric humidity.

After 48 hours, the OD was determined at a measurement wavelength of 660 nm using a Biomek 1000 (Beckmann Instruments GmbH, Munich). The amount of lysine produced was determined with an amino acid analyser from the Eppendorf-BioTronik Co. (Hamburg, Germany) by ion exchange chromatography and post-column derivatisation with ninhydrin detection.

Table 1 gives the results of the trial.

TABLE 1

| | | Lysine-HCl |
|---|---|---|
| Strain | OD (660) | g/l |
| DSM5715::pCR2.1zwa2int | 12.7 | 12.29 |
| DSM5715 | 13.1 | 9.54 |

What is claimed is:

1. An isolated polynucleotide containing a polynucleotide sequence selected from the group consisting of
a) a polynucleotide which is at least 70% identical to a polynucleotide which encodes a polypeptide which contains the amino acid sequence shown in SEQ ID NO:2,
b) a polynucleotide which encodes a polypeptide which contains an amino acid sequence which is at least 70% identical to the amino acid sequence shown in SEQ ID NO:2,
c) a polynucleotide which is complementary to the polynucleotides in a) or b), and
d) a polynucleotide containing at least 15 nucleotides in sequence from the polynucleotide sequence in a), b) or c).

2. The polynucleotide in accordance with claim 1, wherein the polynucleotide is a DNA which is replicatable in coryneform bacteria.

3. The polynucleotide in accordance with claim 2 that is recombinant.

4. The polynucleotide in accordance with claim 1, wherein the polynucleotide is an RNA.

5. The polynucleotide in accordance with claim 2, that comprises the nucleic acid sequence shown in SEQ ID NO:1.

6. Replicatable DNA in accordance with claim 2 containing
(i) the nucleotide sequence shown in SEQ ID NO:1, or
(ii) at least one sequence which corresponds to the sequence (i) within the region of degeneration of the genetic code, or
(iii) at least one sequence, which hybridises with the sequences complementary to sequences (i) or (ii), and optionally
(iv) functionally neutral sense mutations in (i).

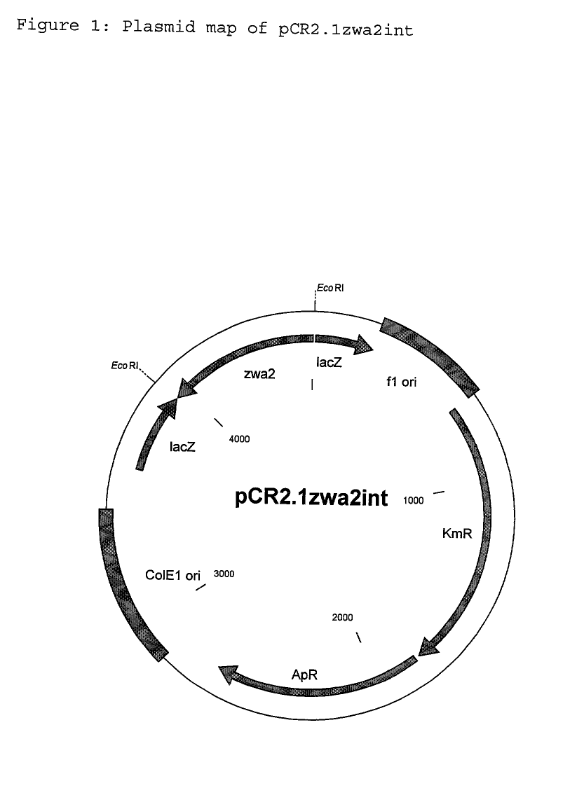

7. A vector having the restriction map given in FIG. 1.

8. The vector according to claim 7, designated as shuttle vector pCR2.1zwa2int, deposited in E. coli DH5a under the name DSM 13113.

9. Coryneform bacteria obtained by integration mutagenesis with the vector in accordance with claim 6.

10. A process for preparing L-amino acids, in particular L-lysine, comprising the following steps:
a) fermentation of the bacteria which produce the required L-amino acid in which at least the zwa2 gene is attenuated,
b) enrichment of the required product in the medium or in the cells of the bacteria, and
c) isolation of the L-amino acid.

11. The process in accordance with claim 10, wherein bacteria are used in which in addition other genes in the biosynthetic pathway for the required L-amino acid in particular the zwa1 gene, are enhanced.

12. The process in accordance with claim 10, wherein bacteria are used in which the metabolic pathways which reduce the formation of the required L-amino acid are at least partly switched off.

13. The process in accordance with claim 10, wherein expression of the polynucleotide which encodes the zwa2 gene is reduced.

14. The process in accordance with claim 10, wherein catalytic properties of the polypeptide (enzyme protein) which encodes the polynucleotide zwa2 are reduced.

15. The process according to claim 10, wherein in order to produce attenuation, the process of integration mutagenesis using the vector pCR2.1zwa2int, shown in FIG. 1 and deposited in E.coli as DSM 13113, is used.

16. The process in accordance with claim 10, wherein to produce L-lysine, bacteria are fermented in which one or more genes selected from the group consisting of
a) the dapA gene encoding dihydrodipicolinate synthase,
b) the lysc gene encoding a feed back resistant aspartate kinase,
c) the pyc gene encoding pyruvate carboxylase,
d) the dapD gene encoding tetradihydrodipicolinate succinylase,
e) the dapE gene encoding succinyldiaminopimelate desuccinylase,
f) the gap gene encoding glyceraldehyde-3-phosphate dehydrogenase,
g) the mqo gene encoding malate:quinone oxidoreductase, and
h) the lysE gene encoding lysine export, are simultaneously enhanced.

17. The process according to claim 16, wherein said gene(s) is enhanced by overexpression or amplification.

18. The process in accordance with claim 10, wherein for the production of L-lysine, bacteria are fermented in which one or more of the genes selected from the group consisting of
a) the pck gene encoding phosphoenolpyruvate carboxykinase, and
b) the pgi gene encoding glucose-6-phosphate isomerase are simultaneously attenuated.

19. The process in accordance with one of claims 10-18, wherein microorganisms from the genus Corynebacterium glutamicum are used.

20. A method for isolating cNDA which encodes a Zwa2 gene product comprising using a polynucleotide sequence in accordance with claim 1 or a portion thereof as a hybridisation probe.

21. A method for isolating cDNA or genes which have a high similarity to the sequence in the Zwa2 gene comprising using a polynucleotide sequence in accordance with claim 1 or a portion thereof as a hybridisation probe.
